(19) United States
(12) Patent Application Publication
(10) Pub. No.: US 2020/0005893 A1
(43) Pub. Date: Jan. 2, 2020

(54) EXTRACTING RELATED MEDICAL INFORMATION FROM DIFFERENT DATA SOURCES FOR AUTOMATED GENERATION OF PROGNOSIS, DIAGNOSIS, AND PREDISPOSITION INFORMATION IN CASE SUMMARY

(71) Applicant: International Business Machines Corporation, Armonk, NY (US)

(72) Inventors: Claudia S. Huettner, Jamaica Plain, MA (US); Jia Xu, Somerville, MA (US); Cheryl L. Eifert, Watertown, MA (US); Vanessa Michelini, Boca Raton, FL (US); Fang Wang, Plano, TX (US); Marta Sanchez-Martin, Somerville, MA (US); Elinor Dehan, Haniel (IR)

(21) Appl. No.: 16/371,204

(22) Filed: Apr. 1, 2019

Related U.S. Application Data
(60) Provisional application No. 62/691,153, filed on Jun. 28, 2018.

(51) Int. Cl. G16B 20/50; G16B 30/00; G16B 50/10; G16B 20/20; C12Q 1/6886; G16H 50/30; G16H 50/20

(52) U.S. Cl. CPC G16B 20/50; G16B 30/00; G16B 50/10; G16H 50/20; C12Q 1/6886; G16H 50/30; G16B 20/20

(57) ABSTRACT

According to embodiments of the present invention, methods, systems and computer readable media are provided for extracting related medical information from various sources to produce a medical evaluation. Genomic information provided from a patient tumor sample is analyzed to determine the presence of one or more mutations in the tumor sample. Hierarchical matching is performed to match the one or more mutations from the patient sample to curated structured data derived from literature. One or more of a prognosis, diagnosis, or predisposition is evaluated based on the matching, wherein the one or more mutations is predictive of a prognosis for a type of tumor, and is a diagnostic marker of a type of tumor. When a pathogenic mutation is detected for a predisposition, a report is generated regarding whether the pathogenic mutation is associated with hereditary cancer.

### CROSS REFERENCE TO RELATED APPLICATION

This application claims priority under 35 USC § 119 from U.S. Provisional Patent Application Ser. No. 62/691,153, entitled Automated Generation of Prognosis, Diagnosis, and Predisposition Information in Case Summary, filed on Jun. 28, 2018, the contents of which are incorporated by reference in their entirety.

### BACKGROUND

### 1. Technical Field

Present invention embodiments relate to extracting related medical information from different data sources for automatically determining relationships between the extracted medical information to generate prognosis, diagnosis and predisposition information.

### 2. Discussion of the Related Art

Various databases exist which contain different types of medical information. In some cases, the databases are not integrated with other databases, making retrieval and assembly of such information challenging and difficult.

### SUMMARY

According to embodiments of the present invention, methods, systems and computer readable media are provided to extract and assemble related medical information, including prognosis, diagnosis and predisposition information. In some aspects, the medical information may be related by a particular mutation, a type of mutation, or a category of mutation.

In some aspects, a patient sample is obtained and analyzed for genetic mutations. A hierarchical matching technique may be used to compare genetic mutations from the patient to curated literature, in order to provide prognosis, diagnosis, and/or predisposition information. A system for extracting related medical information from various sources to produce a medical evaluation is provided herein. Genomic information provided from a patient tumor sample is analyzed via a processor to determine the presence of one or more mutations in the tumor sample. Hierarchical matching is performed via the processor to match the one or more mutations from the patient sample to curated structured data derived from literature. One or more of a prognosis, diagnosis, or predisposition is evaluated based on the matching, wherein the one or more mutations is predictive of a prognosis for a type of tumor and is a diagnostic marker of a type of tumor. When a pathogenic mutation is detected for a predisposition, a report is generated regarding whether the pathogenic mutation is associated with hereditary cancer. Advantages of this approach include integrating complex information, based on genetic commonalities, to determine relationships between prognosis/treatment information, diagnostic information, and predisposition information.

In an embodiment, a cancer-specific ontology is provided, which organizes diseases associated with abnormal cellular proliferation into a plurality of levels from specific categories to broad categories. Hierarchical matching may be applied at a level of a specific category, and when a match is not found, the system may traverse levels of the cancer-specific ontology and reapply the hierarchical matching until a match is found or until the hierarchical matching has been applied to the entire cancer-specific ontology. This approach allows matching to be performed in an optimal manner, with specific matching applied first followed by progressively broader matching.

In another embodiment, the hierarchical matching comprises a first type of matching pertaining to a level of the cancer-specific ontology and a second type of matching pertaining to matching cancer-specific mutations within a level of the ontology. This approach provides a comprehensive strategy to match different types of mutations at each level of the ontology in a hierarchical manner.

In another embodiment, the cancer-specific ontology comprises at least a level comprising specific gene mutations, another level comprising organ-level mutations, and another level comprising solid and blood-borne cancers. This provides a structured, comprehensive approach to analyzing the cancer space to cover all known types of cancer.

In another embodiment, hierarchical matching may determine a mutation based on one or more of matching a specific gene or gene variant, matching a fusion gene, matching based on cancer-specific codon transition bias, matching based on cancer-specific splicing isoforms, or matching based on copy number or gene expression levels. Thus, hierarchical matching may be performed in a manner that identifies a broad range of different types of cancer-specific mutations, to optimize the likelihood that a match will be found by the system.

It is to be understood that the Summary is not intended to identify key or essential features of embodiments of the present disclosure, nor is it intended to be used to limit the scope of the present disclosure. Other features of the present disclosure will become easily comprehensible through the description below.

### BRIEF DESCRIPTION OF THE DRAWINGS

Generally, like reference numerals in the various figures are utilized to designate like components.

FIG. 1 shows an example computing environment for assembling related medical information according to embodiments of the present disclosure.

FIG. 2 is a table for which hierarchical matching may be performed, according to embodiments of the present disclosure.

FIG. 3 is a flowchart showing different levels of a cancer-specific ontology, according to embodiments of the present disclosure.

FIG. 4A is a flowchart showing hierarchical matching from specific to broad matching, according to embodiments of the present disclosure.

FIG. 4B is a flowchart showing hierarchical matching to match specific types of mutations, according to embodiments of the present disclosure.

FIG. 5 shows various categories of cancer-specific mutations, according to embodiments of the present disclosure.

FIG. 6 is a high-level flow chart for providing prognostic information, according to embodiments of the present disclosure.

FIG. 7 is a high-level flow chart for providing diagnostic information, according to embodiments of the present disclosure.

FIG. 8 is a high-level flow chart for providing predisposition information, according to embodiments of the present disclosure.

### DETAILED DESCRIPTION

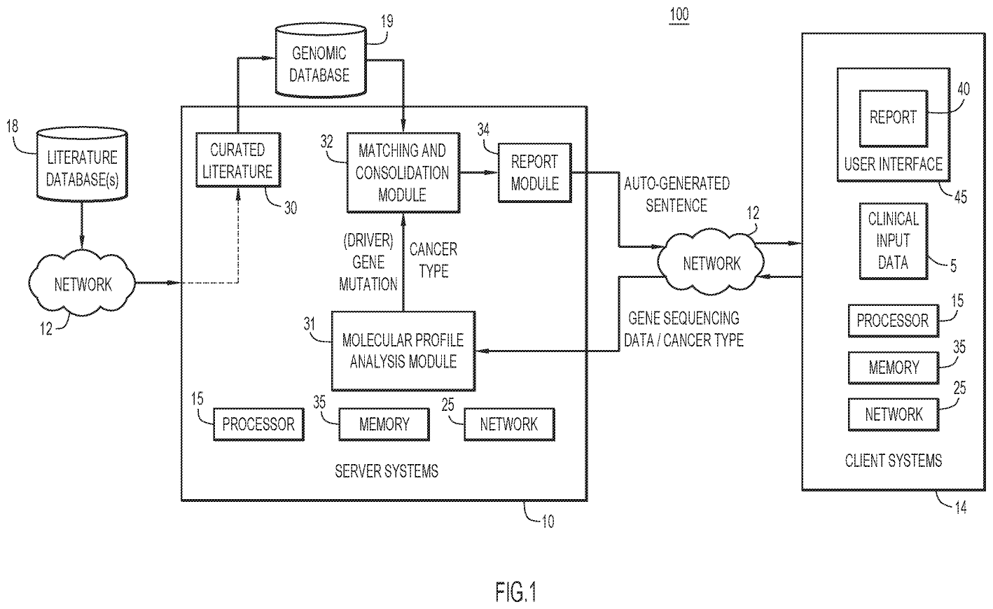

An example environment 100 for use with present invention embodiments is illustrated in FIG. 1. Specifically, the environment includes one or more server systems 10, and one or more client or end-user systems 14. Server systems 10 and client systems 14 may be remote from each other and communicate over a network 12. The network may be implemented by any number of any suitable communications media (e.g., wide area network (WAN), local area network (LAN), Internet, Intranet, etc.). Alternatively, server systems 10 and client systems 14 may be local to each other, and communicate via any appropriate local communication medium (e.g., local area network (LAN), hardwire, wireless link, Intranet, etc.).

Client systems 14 enable users to view reports (e.g., case summaries, genes, gene variants, variant types, condition names, evidence, prognoses, diagnoses/diseases, cancer types, predispositions, mutations (e.g., somatic or germline), treatments, etc.) from server systems 10. The server systems include various modules for analyzing and consolidating information as described herein. A literature database 18 may provide data for analysis that is stored in curated literature 30, and the genomic database 19 may store information from the curated literature 30. In some aspects, curated literature may comprise structured information. In other aspects, curated literature 30 may include information from literature database(s) 18 that has been manually reviewed by a subject matter expert.

Genomic database 19 may contain tables which the matching and consolidation module 32 uses for determining a prognosis, a diagnosis, and/or a predisposition. In some aspects, the genomic database may contain gene names, variant names, variant type information, condition names, evidence, summary information, prognostic information, predisposition information, and diagnostic information. In some aspects, this information may be provided in structured format. Matching and consolidation module 32 may consolidate various types of information for the report (e.g., prognosis, diagnosis, predisposition information, etc.). Matching and consolidation module 32 along with input from molecular profile analysis module 31 may perform hierarchical matching. Report module 34 may generate reports 40 to provide to the user.

The database system may be implemented by any conventional or other database or storage unit, may be local to or remote from server systems 10 and client systems 14, and may communicate via any appropriate communication medium (e.g., local area network (LAN), wide area network (WAN), Internet, hardwire, wireless link, Intranet, etc.). The client systems may present a graphical user (e.g., GUI, etc.) or other user interface 45 (e.g., command line prompts, menu screens, etc.) to solicit information from users pertaining to the desired documents and analysis, and may provide reports 40 including analysis results (e.g., case summaries, genes, gene variants, variant types, condition names, evidence, prognoses, diagnoses/diseases, cancer types, predispositions, mutations (e.g., somatic or germline), treatments, etc.).

Server systems 10 and client systems 14 may be implemented by any conventional or other computer systems preferably equipped with a display or monitor, a base (e.g., including at least one processor 15, one or more memories 35 and/or internal or external network interfaces or communications devices 25 (e.g., modem, network cards, etc.)), optional input devices (e.g., a keyboard, mouse or other input device), and any commercially available and custom software (e.g., server/communications software, molecular profile analysis module 31, matching and consolidation module 32, report module 34, browser/interface software, etc.).

Alternatively, one or more client systems 14 may generate reports when operating as a stand-alone unit. In a stand-alone mode of operation, the client system stores or has access to the data (e.g., literature database 18, clinical input data 5, genomic database 19, etc.), and includes molecular profile analysis module 31 and matching and consolidation module 32 to perform molecule profiling analysis and to match and consolidate data to generate reports. The graphical user (e.g., GUI, etc.) or other interface (e.g., command line prompts, menu screens, etc.) may solicit information from a corresponding user pertaining to the desired documents and analysis, and may provide reports including analysis results.

Server 10 may include one or more modules or units to perform the various functions of present invention embodiments described herein. The various modules (e.g., molecular profile analysis module 31, and matching and consolidation module 32, report module 34, etc.) may be implemented by any combination of any quantity of software and/or hardware modules or units, and may reside within memory 35 of the server and/or client systems for execution by processor 15.

Clinical input data 5 may comprise patient gene sequences, e.g., tumor sequences in a VCF format, which may be analyzed by the molecular profile analysis module 31 to identify driver gene mutations. A listing of driver gene mutations may be provided to the molecular profile analysis module 31 and may be derived from any suitable source (e.g., literature, cancer databases such as The Cancer Genome Atlas, exome sequencing information, etc.). The listing of driver gene mutations may be curated (e.g., manually or in an automated manner) prior to providing to the molecular profile analysis module 31, and may be stored in any suitable database. Cancer cells may have thousands of mutations, and a patient may have different metastases with different mutations. Mutations in driver genes may be common or similar across these different metastases, and are targets for drug development and cancer treatment.

The matching and consolidation module 32 may comprise hierarchical matching techniques as described herein to associate genetic information obtained from a patient with curated literature 30, which may be stored as tables in genomic database 19, to provide prognostic, diagnostic, and predisposition information as described herein. In some aspects, structured data from curated literature 30 may be cached in memory for faster access and sharing.

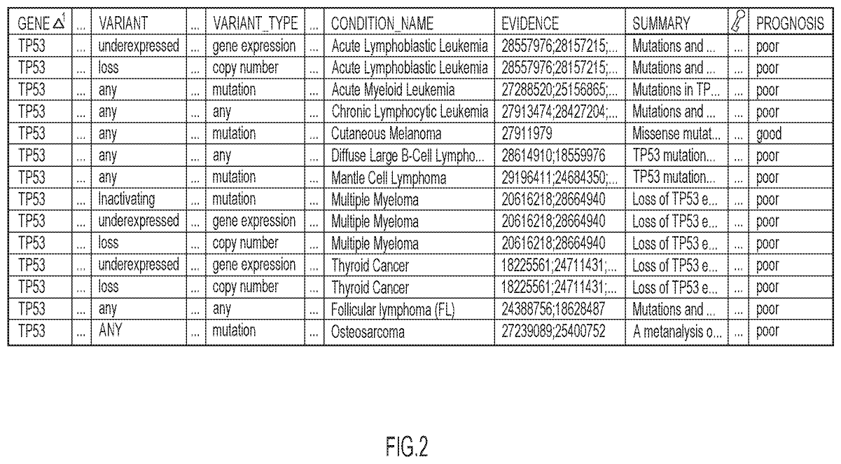

Report 40 may comprise the following prognostic, diagnostic and/or predisposition information, and may be generated by report module 34 and transmitted via network 12 to client systems 14. An embodiment may provide only prognosis, only diagnosis, only predisposition, or any combination thereof, and the table of FIG. 2 may be adjusted accordingly to include or remove columns of information provided in the report to the user based on the type of information requested. The information may be provided as part of a single table (e.g., containing prognostic, diagnostic, or predisposition information) or as multiple tables (e.g., a table for prognostic information, another table for diagnostic information, and yet another table for predisposition information, etc.).

For prognostic data, the report may provide a prognosis relative to a disease. In the case of cancer, the report may provide a prognosis based on the specific genetic mutation(s) identified within the patient's cancer. In some cases, the specific genetic mutation(s) may be described as affecting prognosis in the patient's tumor type. This may be documented in a database, e.g., in a subdirectory labeled prognosis. In this case, the report may include an example sentence stating that: “[mutation type] of gene A is a predictor of [value] prognosis in [cancer type]” as generated by the system. The extracted relationships from literature may additionally quantify the prognosis as poor, good, controversial, or intermediate value levels.

For diagnostic data, the report may link identified genetic mutations to diseases. For example, if a specific genetic alteration is identified, the system will perform an analysis to determine whether the mutation is known to be associated with (considered a hallmark of) or diagnostic of a specific cancer type. In this case, the report 40 may include an example sentence stating that: “[mutation] is a diagnostic marker for [cancer type]”. Unlike prognosis, diagnosis information only shows one level of correlation between a gene/mutation and a cancer type.

For predisposition data, the report may include links between specific genetic alterations that have been associated with a predisposition to a disease, such as hereditary cancer syndromes. Also, for the predisposition table, entries for somatic and germline gene sequencing data may be present. Generally, two scenarios may be considered. For the first scenario, a tumor-only sample does not distinguish a germline mutation from a somatic mutation. In this case, the following example sentences may be generated by the system: “A pathogenic mutation in the [name] gene has been detected. Pathogenic germline mutations in [gene name] have been associated with hereditary cancer.”

For the second scenario, normal or non-tumor DNA and tumor DNA from the patient may be both provided and it may be possible to determine if a genetic alteration is present in germline DNA. For germline mutations, the system may provide an example sentence and report that: “A pathogenic germline mutation in the [GeneName] gene has been detected. Pathogenic germline mutations in [GeneName] have been associated with hereditary cancer.” If mutations are found in more than one gene, the system may provide an example sentence and report that: “Pathogenic germline mutations in [GeneName1], [GeneName2] . . . and [GeneNameN] have been detected. Pathogenic germline mutations in this gene have been associated with hereditary cancer.”

Thus, the system may include a variety of templates to provide diagnostic, prognostic and predisposition data to a patient, based upon hierarchical matching of patient specific molecular data with curated literature 30 (e.g., structured data).

In other aspects, the report may additionally include information about whether or not the mutation is pathogenic, whether or not the mutation is associated with resistance to a drug, a list of drugs associated with treatment of the mutation(s), a list of clinical trials and locations associated with the mutation(s), etc.

If a therapy/treatment for the type of mutation or cancer has been approved by a regulatory agency, the system may provide information about approved treatments. Alternatively, the system may provide clinical trials and locations, in cases in which an approved treatment is not available or has low efficacy. In some cases, the report may contain an annotated sequence listing corresponding to the tumor, listing the specific mutations as determined by the molecular profile analysis module, and associated knowledge regarding prognosis, diagnosis, predisposition, treatment options, etc. The treatments may be ranked, e.g., in order of efficacy based on the specific mutation.

Additionally, clinical input data 5 may be analyzed and may be compared to a physician's diagnosis regarding the type of cancer, and in some cases, the system may validate the physician's diagnosis of the type of cancer.

Curated literature 30 may be generated manually or semi-automatically (e.g., using machine learning and/or natural language processing) from analysis of the literature database(s) 18. Typical structured data for curated literature 30 may be obtained as follows. Each gene mutation may be referred to as a biomarker. Every biomarker may be described by a combination of gene/variant_type/variant. The various combinations of biomarker and cancer type (referred to as condition_name in FIG. 2) produce different prognosis value levels (as shown in the last column of FIG. 2), some of the combinations are at a specific mutation (such as KRAS G13D), some of the combinations are at a more intermediate level (such as KRAS codon 12, TP53 inactivating mutations), and some of the combinations are at very large scope (such as TP53 any variant, any variant type and KRAS any mutation).

FIG. 2 shows an example table schema for prognosis, designed to facilitate hierarchical processing according to the techniques provided herein. However, the table may be modified to include information for diagnosis or for predisposition, e.g., obtained from analysis of literature database(s) 18, etc. In addition, in some cases, the system may provide treatment information associated with geolocation information, regarding nearby clinical trials or other treatment services.

For a given patient's gene sequencing data, molecular profile analysis module 31 may analyze the gene sequencing data to obtain a list of the driver genes with pathogenic/vus mutations. For each driver gene mutation, matching and consolidation module 32 compares (e.g., using hierarchical matching) mutation data to the curated literature, stored in genomic database 19, to determine if there is a match. The matching and consolidation module 32 has a hierarchical progression starting from the smallest scope at a specific mutation progressing to a large scope. For example, if a match is not found at the specific mutation level, then the matching scope is gradually enlarged until a match is found or the system determines that no relevant entry is found. Matching and consolidation module 32 may also perform a cancer type progression, from specific/relevant cancers through parent/child relationships in cancer ontology, cancer categories (solid/hematological) and to the largest scope for any cancer.

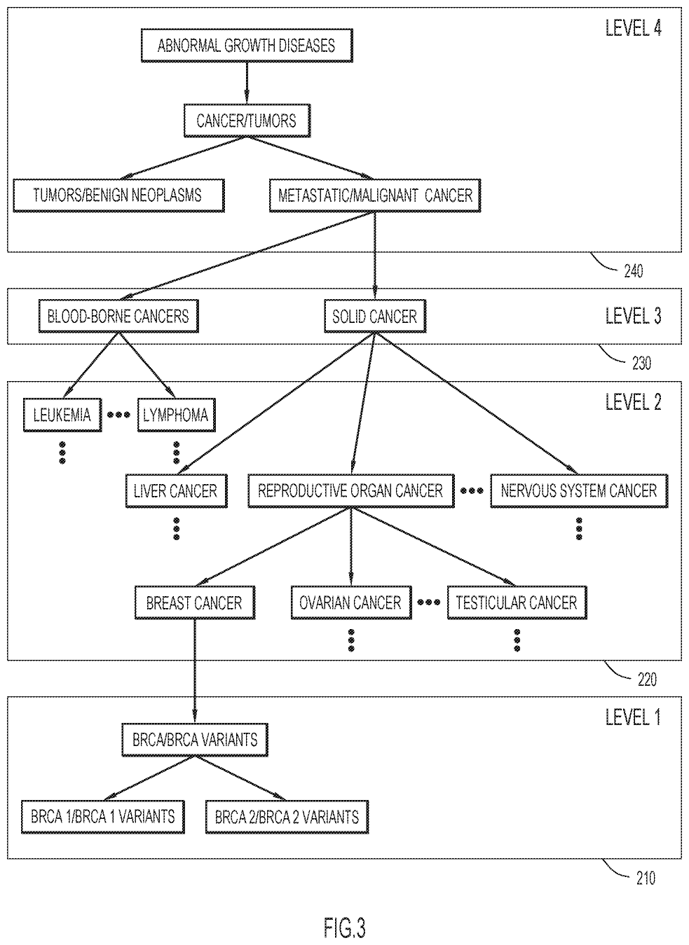

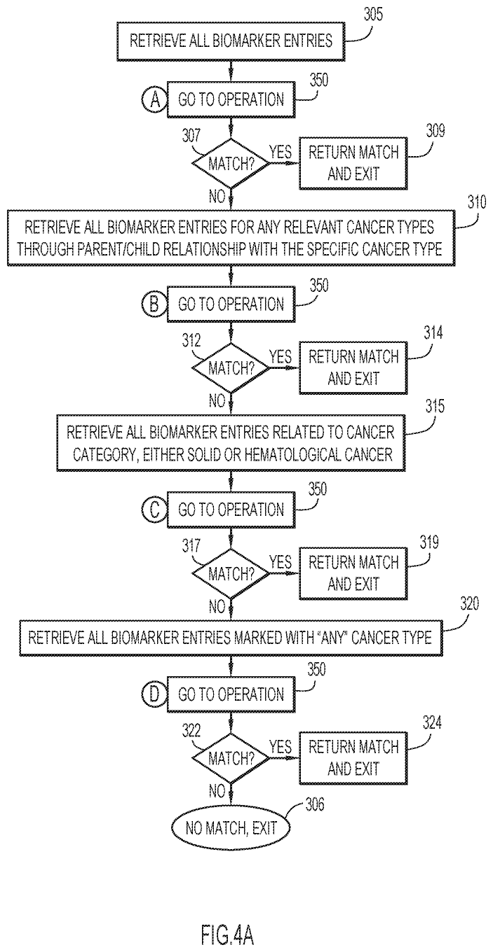

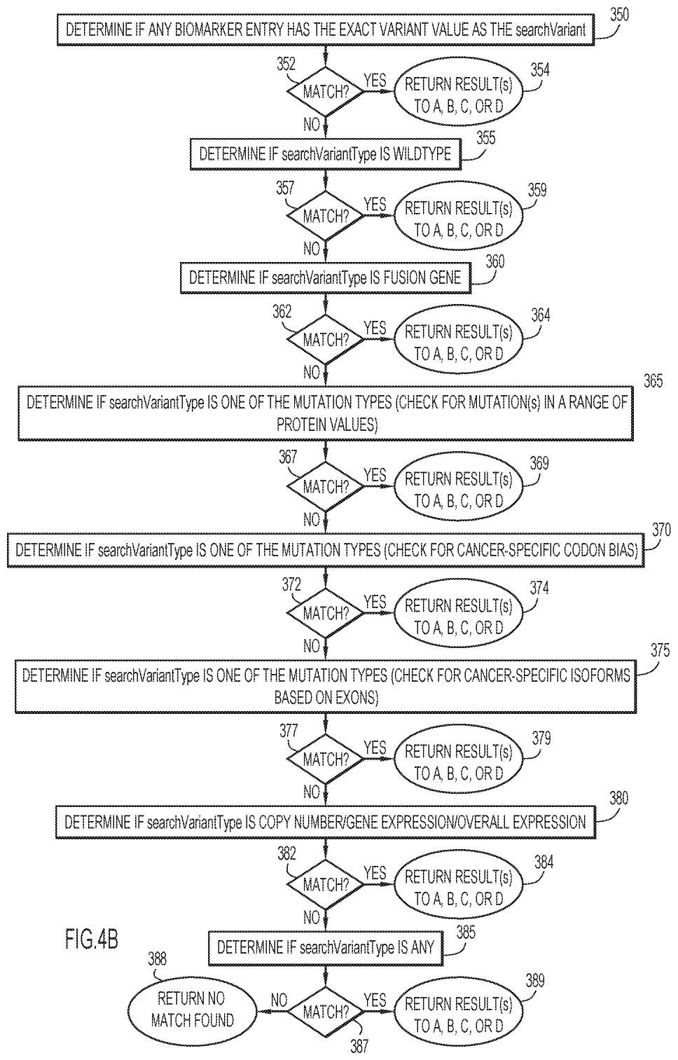

FIG. 3 shows an example ontology/categorization for cancer which may be used with FIGS. 4A-4B. Other ontologies for cancer are included within the scope of this discussion. Layers may be added, removed or combined with respect to the example ontology provided herein. With reference to the operations above, these operations may be applied to various layers of the example ontology.

For example, the matching and consolidation module may retrieve specific biomarkers from level 1 shown as block 210 (see, FIG. 3), and may search the retrieved biomarkers to determine if there is a match with the patient sample. If a match is found, a result is returned.

If a match is not found, the matching and consolidation module moves up one level and retrieves biomarkers for a parent type of cancer as shown in level 2 shown as block 220. Here, parent/child relationships may be considered. For example, a parent relationship for the breast cancer category may include reproductive organ cancer. In level 2, parent biomarkers (and corresponding subcategories) are searched to determine if there is a match with the patient sample. If a match is found, a result is returned.

If a match is not found, the matching and consolidation module moves up one level and retrieves biomarkers for broader categories of cancer, covering solid and blood based diseases) in level 3 shown as block 230. If a match with the patient sample is found, a result is returned.

If a match is not found, the matching and consolidation module continues to traverse levels of the ontology and to retrieve biomarkers from level 4 shown as block 240. If a match is found, a result is returned. Otherwise, the system reports no match, once the top of the ontology has been reached.

In general, the system starts at a specific level, and traverses the ontology to progressively broader levels in order to determine a match. Whenever the system moves up a level, biomarkers within that level (and corresponding lower levels) may be evaluated (e.g., breast cancer may include all BRCA genes and variants; reproductive organ cancer may include breast, ovarian and testicular cancer, etc.; and solid cancer may include all types of solid cancer, etc.).

In some aspects, the matching and consolidation module 32 may progressively match in a matching procession, beginning with a small scope (e.g., matching a specific mutation) to a broad scope (e.g., a category of cancer). Operations 305-324, as shown in FIG. 4A, show a hierarchical matching strategy, wherein the matching progresses from specific matching to broad matching. For each given gene variant from the patient profile (referred to as search Variant), the following four operations may be performed in sequence to match an entry in the table:
At operation 305, for the specific cancer type, retrieve all biomarker entries. Perform searchByCancerType algorithm at operation 350 (see, FIG. 4B) and determine if there is a match at operation 307. If yes, return the match (to be used by report module 34 to auto-generate the report sentence) and exit at operation 309. If not, continue to operation 310.
At operation 310, for any relevant cancer types through parent/child relationship with the specific cancer type, retrieve all biomarker entries. Perform searchByCancerType algorithm at operation 350 and determine if there is a match at operation 312. If yes, return the match (to be used by report module 34 to auto-generate the report sentence) and exit at operation 314. If not, continue to operation 315.
At operation 315, for the corresponding cancer category (either solid or hematological), retrieve all biomarker entries related to either solid or hematological cancer category. Perform searchByCancerType algorithm at operation 350 and determine if there is a match at operation 317. If yes, return the match (to be used by report module 34 to auto-generate the report sentence) and exit at operation 319. If not, continue to operation 320.
At operation 320, for cancer type=“any”, retrieve all biomarker entries marked with “any” cancer type. Perform searchByCancerType algorithm at operation 350 and determine if there is a match at operation 322. If yes, return the match (to be used by report module 34 to auto-generate the report sentence) and exit at operation 324. If not, report no match at operation 306.

After each of operations 305-320, the searchByCancerType technique provided below may be performed. Operations 350-389 show aspects of the searchByCancerType technique, as shown in FIG. 4B, which uses a matching strategy to match a type of mutation. This technique may use part or all of the genetic or proteomic information provided from the patient sample to determine whether a match is found using information from cancer databases. In some aspects, genomic information from the patient is translated into proteomic information to facilitate biomarker analysis.

This approach may first filter out wildtype and genetic variants (normal biomarkers, before testing for the presence of cancer-specific biomarkers). Cancer-specific matching may include searching for cancer-specific fusion genes/proteins, which may include classes of oncogenes that are specific to tumor/cancer cells. In this case, cancer cells may exhibit genomic instability, leading to the rearrangement of the genome inside the cell, resulting in fusion genes that produce fusion proteins. Fusion genes may be found in a wide variety of cancer types including adenoid cystic carcinoma, breast carcinoma, Ewing sarcoma, synovial sarcoma, glioblastoma multiforme, lung cancer, clear cell renal cell carcinoma, bladder cancer, prostate cancer, ovarian cancer, colorectal cancer, etc. Accordingly, the searching technique determines whether the patient sample matches known fusion biomarkers.

If fusion biomarkers are not identified, then the system may search for various other types of mutations. This may include searching specific ranges of a protein for one or more mutations, searching for codon-based mutations (e.g., presenting as cancer-specific codon transition bias), and cancer-specific isoforms. Cancer cells may have somatic mutations at specific locations (e.g., point mutations). This may include specific codon mutations, in which codons are mutated in a manner that is prevalent in cancer cells as compared to normal cells, referred to as codon transition bias. Cancer cells may also have specific splicing isoforms (e.g., in which expressed exons are arranged, inserted or deleted in a manner found in cancer cells).

In some cases, the cancer-specific ontology may include biomarkers, which have designations corresponding to variant and variant type as provided below. The searchByCancerType technique may perform the following operations in sequence as shown in FIG. 4B:
The hierarchical matching techniques provided herein operate based on FIGS. 4A-4B. Specifically, at ‘A’, ‘B’, ‘C’, and ‘D’ in FIG. 4A, the system proceeds to operation 350 in FIG. 4B. When a match is found, the system returns to a corresponding match in FIG. 4A. For example, if operation 350 originated from ‘A’, then match 352 will return to match 307 immediately following ‘A’. If operation 350 originated from ‘B’, then match 352 will return to match 312 immediately following ‘B’, and so forth.
At operation 350, matching and consolidation module 32 (e.g., using a dataCheck function) determines if any biomarker entry has the exact variant value as the searchVariant, which is determined by searching a genomic database for exact matches. Variant values may be determined based on an association with a cancer type, presence of matching codons, presence of different amino acid substitutions, etc. The variant values may be standardized to be in the same format as the values in the database table for matching. At operation 352, the system determines if there is a match. If there is only one match, results are returned at operation 354. If there are multiple matches, the closest cancer type is selected and returned at operation 354. If there is no match, the system continues to operation 355.
At operation 355, the system determines if searchVariantType is wildtype, by checking if any biomarker entry has the variant type=“wildtype” and variant=“any” or variant matches searchVariant. At operation 357, the system determines if there is a match. If there is only one match, the results are returned at operation 359. If there are multiple matches, the closest cancer type is selected and returned at operation 359. If there is no match, the system continues to operation 360.
At operation 360, the system determines if searchVariantType is fusion gene (e.g., a hybrid gene, which is formed from two previously separate genes—fusion genes may occur as a result of: translocation, interstitial deletion, chromosomal inversion, etc.), by checking if any biomarker entry has the variant type =“fusion gene” and variant matches searchVariant. At operation 362, the system determines if there is a match. If there is only one match, the results are returned. If there are multiple matches, the closest cancer type is selected and returned. If there is no match, the system checks if any biomarker entry has the variant type =“fusion gene” and variant =“any”. If there is only one match, the results are returned at operation 364. If there are multiple matches, the closest cancer type is selected and returned at operation 364. If there is no match, the system continues to operation 365.
At operation 365, the system determines if searchVariantType is one of the mutation types, by checking if any biomarker entry with variantType matches searchVariantType or variantType=“mutation” and variant value as range which covers the protein position of searchVariant. At operation 367, the system determines if there is a match. If there is only one match, the results are returned at operation 369. If there are multiple matches, the closest cancer type is selected and returned at operation 369. If there is no match, the system continues to operation 370.
At operation 370, the system determines if searchVariantType is one of the mutation types, by checking if any biomarker entry with variantType matches searchVariantType or variantType=“mutation” and variant value has codon value matches that of searchVariant. At operation 372, the system determines if there is a match. If there is only one match, the results are returned at operation 374. If there are multiple matches, the closest cancer type is selected and returned at operation 374. If there is no match, the system continues to operation 375.
At operation 375, the system determines if searchVariantType is one of the mutation types, by checking if any biomarker entry with variantType matches searchVariantType or variantType=“mutation” and variant value has exon value matches that of searchVariant. At operation 377, the system determines if there is a match. If there is only one match, the results are returned at operation 379. If there are multiple matches, the closest cancer type is selected and returned at operation 379. If there is no match, the system continues to operation 380.
At operation 380, the system checks if searchVariantType is copy number/gene expression/overall expression, and checks if any biomarker entry with variantType matches searchVariantType and variant=“any”. At operation 382, the system determines if there is a match. If there is only one match, the results are returned at operation 384. If there are multiple matches, the closest cancer type is selected and returned at operation 384. If there is no match, the system continues to operation 385.
At operation 385, the system checks if any biomarker entry with variantType=“any” and variant=“any”. At operation 387, the system determines if there is a match. If there is only one match, the results are returned at operation 389. If there are multiple matches, the closest cancer type is selected and returned at operation 389. If there is no match, the system indicates that no match was found at operation 388.

“Pick the closest cancer type” means finding the cancer type through a parent child relationship in the ontology tree with the shortest distance from the diagnosed cancer type of the patient. If there are two cancers with the same distance, the upstream one is selected over the downstream one.

In some cases, the system may utilize machine learning to associate drugs or combinations of drugs with a particular type of cancer.

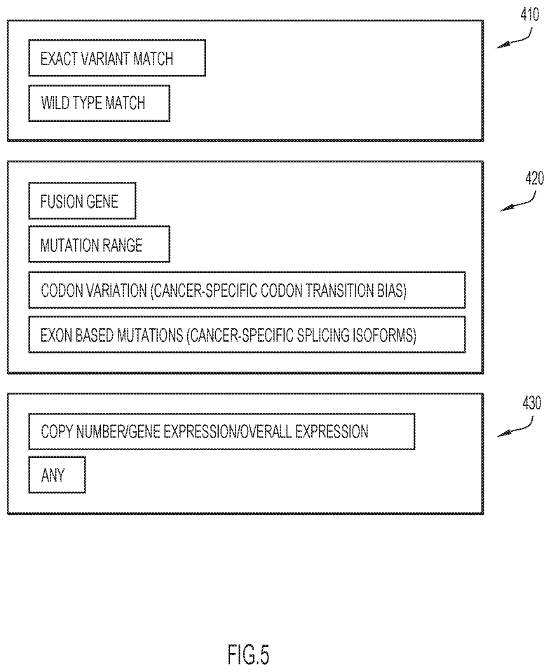

FIG. 5 is an illustration showing various granularities of mutations as well as corresponding wild-type and normal variants (not cancer-specific). Category 410 shows specific matching to a gene or variant, which includes matching specific sequences. This match may be performed initially to screen out wildtype or naturally occurring variants that are not associated with cancer. Category 420 allows matching for different types of cancer-specific mutations, such as fusion genes/proteins resulting from genomic instability of cancer cells, mutations found in cancer, codon variations that have been specifically been shown to occur, usually at higher frequencies, in cancer cells as compared to normal cells (known as codon transition bias), and cancer-specific splicing isoforms—isoforms that may include additions, deletions or other abnormal combinations of exons that are present in cancer cells. Category 430 may include information linking protein expression (of the corresponding gene) or any other type of analysis to cancer.

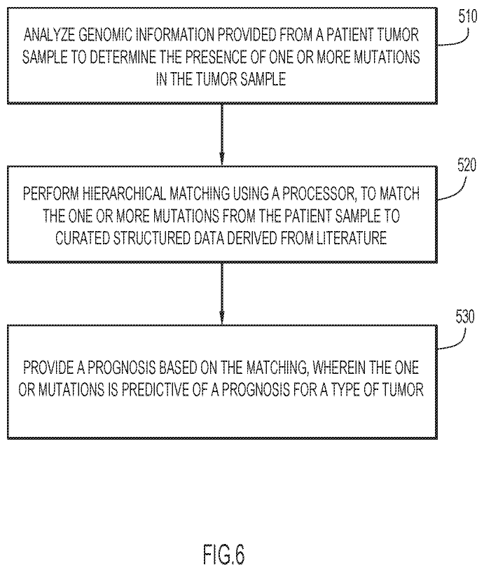

FIG. 6 is a high-level flow chart for providing prognostic information, according to embodiments of the present disclosure. At operation 510, genomic information provided from a patient tumor sample is analyzed to determine the presence of one or more mutations in the tumor sample. At operation 520, hierarchical matching is performed using a processor, to match the one or more mutations from the patient sample to curated structured data derived from literature. At operation 530, a prognosis is provided based on the matching, wherein the one or more mutations is predictive of a prognosis for a type of tumor.

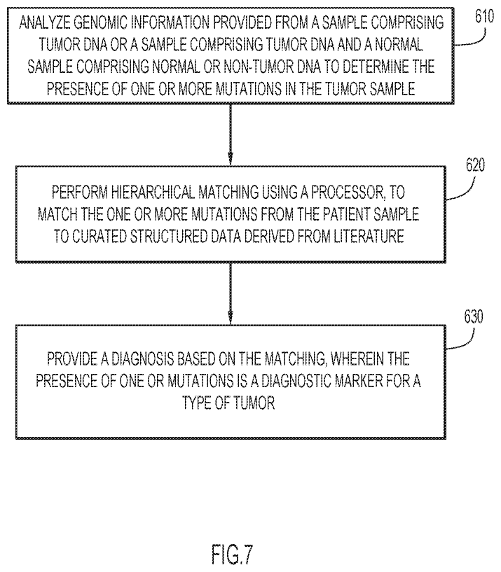

FIG. 7 is a high-level flow chart for providing diagnostic information, according to embodiments of the present disclosure. At operation 610, genomic information provided from a sample comprising tumor DNA or a sample comprising normal or non-tumor DNA is analyzed to determine the presence of one or more mutations in the tumor sample. At operation 620, hierarchical matching is performed using a processor, to match the one or more mutations from the patient sample to curated structured data derived from literature. At operation 630, a diagnosis is provided based on the matching, wherein the one or more mutations is a diagnostic marker for a type of tumor.

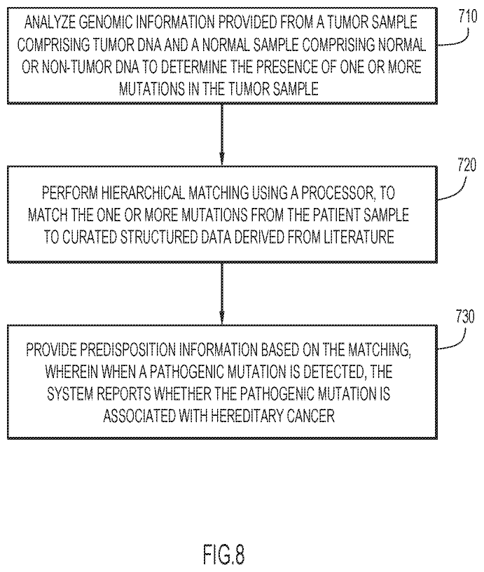

FIG. 8 is a high-level flow chart for providing predisposition information, according to embodiments of the present disclosure. At operation 710, genomic information provided from a patient tumor sample is analyzed to determine the presence of one or more mutations in the tumor sample. At operation 720, hierarchical matching is performed using a processor, to match the one or more mutations from the patient sample to curated structured data derived from literature. At operation 730, predisposition information is provided based on the matching, wherein when a pathogenic mutation is detected, the system reports whether the pathogenic mutation is associated with hereditary cancer. The system may perform any of the operations provided in FIGS. 6-8, or any combination thereof.

Advantages of present techniques include integrating complex information, based on genetic or proteomic commonalities, to determine relationships between prognosis/treatment information, diagnostic information, and predictive information. These approaches allow matching to be performed in an optimal manner, with specific matching applied first followed by broader matching. Present techniques allow for matching different types of mutations within each level of the ontology, and providing a structured, comprehensive approach to analyzing the cancer space. Thus, hierarchical matching may be performed in a manner that identifies a broad range of different types of cancer-specific mutations in a specific manner, to optimize the likelihood that a match will be found by the system. The system also integrates medical data from multiple sources.

It will be appreciated that the embodiments described above and illustrated in the drawings represent only a few of the many ways of implementing embodiments for providing consolidated information to a patient for prognosis, diagnosis and predisposition information. The present embodiments are not limited to cancer, but may apply to any disease or disorder associated with genetic mutations.

The environment of the present invention embodiments may include any number of computer or other processing systems (e.g., client or end-user systems, server systems, etc.) and databases or other repositories arranged in any desired fashion, wherein the present invention embodiments may be applied to any desired type of computing environment (e.g., cloud computing, client-server, network computing, mainframe, stand-alone systems, etc.). The computer or other processing systems employed by the present invention embodiments may be implemented by any number of any personal or other type of computer or processing system (e.g., desktop, laptop, PDA, mobile devices, etc.), and may include any commercially available operating system and any combination of commercially available and custom software (e.g., browser software, communications software, server software, molecular profile analysis module 31, matching and consolidation module 32, report module 34, etc.). These systems may include any types of monitors and input devices (e.g., keyboard, mouse, voice recognition, etc.) to enter and/or view information.

It is to be understood that the software (e.g., molecular profile analysis module 31, matching and consolidation module 32, report module 34, etc.) of the present invention embodiments may be implemented in any desired computer language and could be developed by one of ordinary skill in the computer arts based on the functional descriptions contained in the specification and flow charts illustrated in the drawings. Further, any references herein of software performing various functions generally refer to computer systems or processors performing those functions under software control. The computer systems of the present invention embodiments may alternatively be implemented by any type of hardware and/or other processing circuitry.

The various functions of the computer or other processing systems may be distributed in any manner among any number of software and/or hardware modules or units, processing or computer systems and/or circuitry, where the computer or processing systems may be disposed locally or remotely of each other and communicate via any suitable communications medium (e.g., LAN, WAN, Intranet, Internet, hardwire, modem connection, wireless, etc.). For example, the functions of the present invention embodiments may be distributed in any manner among the various end-user/client and server systems, and/or any other intermediary processing devices. The software and/or algorithms described above and illustrated in the flow charts may be modified in any manner that accomplishes the functions described herein. In addition, the functions in the flow charts or description may be performed in any order that accomplishes a desired operation.

The software of the present invention embodiments (e.g., molecular profile analysis module 31, matching and consolidation module 32, report module 34, etc.) may be available on a non-transitory computer useable medium (e.g., magnetic or optical mediums, magneto-optic mediums, floppy diskettes, CD-ROM, DVD, memory devices, etc.) of a stationary or portable program product apparatus or device for use with stand-alone systems or systems connected by a network or other communications medium.

The communication network may be implemented by any number of any type of communications network (e.g., LAN, WAN, Internet, Intranet, VPN, etc.). The computer or other processing systems of the present invention embodiments may include any conventional or other communications devices to communicate over the network via any conventional or other protocols. The computer or other processing systems may utilize any type of connection (e.g., wired, wireless, etc.) for access to the network. Local communication media may be implemented by any suitable communication media (e.g., local area network (LAN), hardwire, wireless link, Intranet, etc.).

The system may employ any number of any conventional or other databases, data stores or storage structures (e.g., files, databases, data structures, data or other repositories, etc.) to store information (e.g., reports, data extracted from literature, prognostic information, diagnostic information, predisposition information, genomic information, curated literature 30, gene, gene variants, variant types, condition names, evidence, prognoses, diagnoses, predisposition information, etc.). The database system may be implemented by any number of any conventional or other databases, data stores or storage structures (e.g., files, databases, data structures, data or other repositories, etc.) to store information (e.g., reports, data extracted from literature, prognostic information, diagnostic information, predisposition information, genomic information, curated literature 30, gene, gene variants, variant types, condition names, evidence, prognoses, diagnoses, predisposition information, etc.). The database system may be included within or coupled to the server and/or client systems. The database systems and/or storage structures may be remote from or local to the computer or other processing systems, and may store any desired data (e.g., reports, prognostic information, data extracted from literature, diagnostic information, predisposition information, genomic information, curated literature 30, gene, gene variants, variant types, condition names, evidence, prognoses, diagnoses, predisposition information, etc.).

The present invention embodiments may employ any number of any type of user interface (e.g., Graphical User Interface (GUI), command-line, prompt, etc.) for obtaining or providing information (e.g. reports, prognostic information, diagnostic information, predisposition information, genomic information, curated literature 30, gene, gene variants, variant types, condition names, evidence, prognoses, diagnoses, predisposition information, etc.), where the interface may include any information arranged in any fashion. The interface may include any number of any types of input or actuation mechanisms (e.g., buttons, icons, fields, boxes, links, etc.) disposed at any locations to enter/display information and initiate desired actions via any suitable input devices (e.g., mouse, keyboard, etc.). The interface screens may include any suitable actuators (e.g., links, tabs, etc.) to navigate between the screens in any fashion.

The report may include any information arranged in any fashion, and may be configurable based on rules or other criteria to provide desired information to a user (e.g., reports, prognostic information, diagnostic information, predisposition information, genomic information, gene, gene variants, variant types, condition names, evidence, prognoses, diagnoses, predisposition information, etc.).

The present invention embodiments are not limited to the specific tasks or algorithms described above, but may be utilized for any application involving matching genetic information from a biological sample to knowledge in the literature associated with genomic information.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises”, “comprising”, “includes”, “including”, “has”, “have”, “having”, “with” and the like, when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

What is claimed is:

1. A method for extracting related medical information from various sources to produce a medical evaluation comprising:
analyzing, via a processor, genomic information provided from a patient tumor sample to determine the presence of one or more mutations in the tumor sample;
performing hierarchical matching, via the processor, to match the one or more mutations from the patient sample to curated structured data derived from literature; and
evaluating one or more of a prognosis, diagnosis, or predisposition based on the matching, wherein the one or more mutations is predictive of a prognosis for a type of tumor, and is a diagnostic marker of a type of tumor;
wherein when a pathogenic mutation is detected for a predisposition, reporting whether the pathogenic mutation is associated with hereditary cancer.

2. The method of claim 1, further comprising:
providing a cancer-specific ontology, which organizes diseases associated with abnormal cellular proliferation in a plurality of levels from specific categories to broad categories; and
applying the hierarchical matching at a level of the ontology, and when a match is not found, traversing the cancer-specific ontology and reapplying the hierarchical matching until a match is found or until the hierarchical matching has been applied to the entire cancer-specific ontology.

3. The method of claim 2, wherein the hierarchical matching comprises a first type of matching pertaining to a level of the cancer-specific ontology and a second type of matching pertaining to identifying cancer-specific mutations.

4. The method of claim 1, wherein the one or more mutations is a driver mutation.

5. The method of claim 2, wherein the cancer-specific ontology comprises at least a level comprising specific gene mutations, a level comprising organ-based cancers, and a level comprising solid and blood-borne cancers.

6. The method of claim 1, wherein the genomic information from the patient is translated into proteomic information for biomarker analysis.

7. The method of claim 1, wherein hierarchical matching to determine a mutation may include one or more of matching a fusion biomarker, matching based on cancer-specific codon transition bias, matching based on cancer-specific splicing isoforms, or matching based on copy number or gene expression levels.

8. A system for extracting related medical information from various sources to produce a medical evaluation, wherein the system comprises at least one processor configured to:
analyze genomic information provided from a patient tumor sample to determine the presence of one or more mutations in the tumor sample;
perform hierarchical matching to match the one or more mutations from the patient sample to curated structured data derived from literature; and
evaluate one or more of a prognosis, diagnosis, or predisposition based on the matching, wherein the one or more mutations is predictive of a prognosis for a type of tumor, and is a diagnostic marker of a type of tumor;
wherein when a pathogenic mutation is detected for a predisposition, reporting whether the pathogenic mutation is associated with hereditary cancer.

9. The system of claim 8, wherein the at least one processor is configured to:
provide a cancer-specific ontology, which organizes diseases associated with abnormal cellular proliferation in a plurality of levels from specific categories to broad categories; and
apply the hierarchical matching at a level of the ontology, and when a match is not found, traversing the cancer-specific ontology and reapplying the hierarchical matching until a match is found or until the hierarchical matching has been applied to the entire cancer-specific ontology.

10. The system of claim 9, wherein the hierarchical matching comprises a first type of matching pertaining to a level of the cancer-specific ontology and a second type of matching pertaining to identifying cancer-specific mutations.

11. The system of claim 8, wherein the one or more mutations is a driver mutation.

12. The system of claim 9, wherein the cancer-specific ontology comprises at least a level comprising specific gene mutations, a level comprising organ-based cancers, and a level comprising solid and blood-borne cancers.

13. The system of claim 8, wherein the genomic information from the patient is translated into proteomic information for biomarker analysis.

14. The system of claim 8, wherein hierarchical matching to determine a mutation may include one or more of matching a fusion biomarker, matching based on cancer-specific codon transition bias, matching based on cancer-specific splicing isoforms, or matching based on copy number or gene expression levels.

15. A computer program product for extracting related medical information from various sources to produce a medical evaluation, the computer program product comprising a computer readable storage medium having program instructions embodied therewith, the program instructions executable by a computer to cause the computer to:
analyze genomic information via a processor provided from a patient tumor sample to determine the presence of one or more mutations in the tumor sample;
perform hierarchical matching via the processor, to match the one or more mutations from the patient sample to curated structured data derived from literature; and
evaluate one or more of a prognosis, diagnosis, or predisposition, wherein based on the matching, wherein the one or more mutations is predictive of a prognosis for a type of tumor, and is a diagnostic marker of a type of tumor;
wherein when a pathogenic mutation is detected for a predisposition, reporting whether the pathogenic mutation is associated with hereditary cancer.

16. The computer program product of claim 15, wherein the instructions are further executable by the computer to cause the computer to:
provide a cancer-specific ontology, which organizes diseases associated with abnormal cellular proliferation in a plurality of levels from specific categories to broad categories; and
apply the hierarchical matching at a level of the ontology, and when a match is not found, traversing the cancer-specific ontology and reapplying the hierarchical matching until a match is found or until the hierarchical matching has been applied to the entire cancer-specific ontology.

17. The computer program product of claim 16, wherein the hierarchical matching comprises a first type of matching pertaining to a level of the cancer-specific ontology and a second type of matching pertaining to identifying cancer-specific mutations.

18. The computer program product of claim 15, wherein the one or more mutations is a driver mutation.

19. The computer program product of claim 15, wherein the genomic information from the patient may be translated into proteomic information for biomarker analysis.

20. The computer program product of claim 16, wherein hierarchical matching to determine a mutation may include one or more of matching a fusion biomarker, matching a sequence comprising cancer-specific codon transition bias, matching cancer-specific splicing isoforms, or matching based on copy number or gene expression levels.
